(19) United States
(12) Patent Application Publication
(10) Pub. No.: US 2016/0302766 A1
(43) Pub. Date: Oct. 20, 2016

(54) ULTRASONIC PROBE AND ULTRASONIC IMAGING SYSTEM COMPRISING THE SAME

(71) Applicant: SHENZHEN MINDRAY BIO-MEDICAL ELECTRONICS CO., LTD., SHENZHEN (CN)

(72) Inventors: Jianhui LIU, Shenzhen (CN); Yong LI, Shenzhen (CN); Shuo LIU, Shenzhen (CN); Qun LIN, Shenzhen (CN)

(73) Assignee: SHENZHEN MINDRAY BIO-MEDICAL ELECTRONICS CO., LTD.

(21) Appl. No.: 15/197,474

(22) Filed: Jun. 29, 2016

Related U.S. Application Data
(63) Continuation of application No. PCT/CN2014/077331, filed on May 13, 2014.

(30) Foreign Application Priority Data
Dec. 31, 2013 (CN) 201310754772.X

(51) Int. Cl. A61B 8/00

(52) U.S. Cl. CPC A61B 8/4455; A61B 8/4209; A61B 8/54

(57) ABSTRACT

An ultrasonic probe and an ultrasonic imaging system including the ultrasonic probe are provided. The ultrasonic probe comprises a probe body, an elastic liner, an adjusting assembly and a fixing member. The ultrasonic probe can control an elastic adjustment base to squeeze or loosen the elastic liner by tightening or loosening an adjusting rotary knob, and thereby the insertion depth of the probe body in the elastic liner can be adjusted by tightening or loosening the probe body. The angle of the ultrasonic waves and the pressurization depth of the probe body of the ultrasonic probe can be adjusted, and the ultrasonic probe can be secured to a target object for a long time for continuous ultrasonic imaging. Therefore, the ultrasonic probe can be widely applied to a clinical occasion where long-time continuous ultrasonic monitoring is required.

### TECHNICAL FIELD

The present disclosure relates to medical imaging, particularly to an ultrasonic probe and an ultrasonic imaging system comprising the ultrasonic probe.

### BACKGROUND

An ultrasonic imaging technology is widely applied to human tissue imaging and pathological diagnosis. An ultrasonic imaging system is an instrument for detecting a human body through ultrasonic waves so as to obtain a human tissue image. Ultrasonic waves are generated and ultrasonic echoes are received by an ultrasonic probe, which has a very important effect on a detection effect. Different ultrasonic probes are used for different imaging regions. The probes also have different sizes, shapes and operating frequencies in accordance with different ages, figures and positions of scanned tissues and organs of patients.

A traditional body surface medical ultrasonic probe generally has a non-fixed structure, and is handheld by a doctor during use and pressed on a detected position of a detected person. The ultrasonic probe is rotated, moved or pressurized according to imaging needs to obtain a required ultrasonic image. However, the ultrasonic probe is secured to a certain position of the human body for a long time during examination of some diseases. The traditional ultrasonic probe with the non-fixed structure has difficulty in adapting to such use due to the increase of labor intensity and waste of human power caused by using the doctor's hands to secure the ultrasonic probe to a certain position of the human body for a long time. Such use also influences a detection effect due to the inevitable changes in the handheld position.

### SUMMARY

Therefore, there is a need for providing (i) a fixed ultrasonic probe capable of adjusting an angle and a pressurization depth and (ii) an ultrasonic imaging system comprising the ultrasonic probe.

In some embodiments, an ultrasonic probe is provided. The ultrasonic probe may include: a probe body which may be used to ultrasonically scan a target object; an elastic liner surrounding the probe body, where the probe body may pass through the elastic liner and be movable relative to the elastic liner; an adjusting assembly provided with a containing cavity, where the elastic liner may be arranged in the containing cavity and be able to be abutted against a cavity wall of the containing cavity, and where the probe body may be tightened or loosened by the adjusting assembly by tightening or loosening the elastic liner so as to adjust a position of the probe body with respect to the elastic liner; and a fixing member fixedly connected with the adjusting assembly, where the fixing member may be used to for securing to the target object.

In some embodiments, an ultrasonic imaging system is provided. The ultrasonic imaging system may include the ultrasonic probe described above.

### DESCRIPTION OF THE DRAWINGS

FIG. 1 is a schematic structural view of an ultrasonic probe of one embodiment;

FIG. 2 is a sectional view of the ultrasonic probe in FIG. 1;

FIG. 3 is an exploded view of the ultrasonic probe in FIG. 1;

FIG. 4 schematically shows a matching of the probe body with the elastic liner in FIG. 1; and

FIG. 5 is an exploded view of the probe body and the elastic liner in FIG. 4.

### DETAILED DESCRIPTION

For the convenience of understanding the present disclosure, an ultrasonic probe and an ultrasonic imaging system including the ultrasonic probe of the present disclosure will be described below more comprehensively with reference to relevant drawings. Some embodiments of the present disclosure are given in the drawings. However, the present disclosure may be achieved in many different forms and will not be limited to the embodiments described herein. On the contrary, the purpose of providing the embodiments is to understand the present disclosure more thoroughly and comprehensively.

It should be noted that when an element is described as “being secured to” another element, the element may be directly secured on another element, or an intermediate element may also exist. When an element is described as “being connected with” another element, the element may be directly connected to another element, or an intermediate element may also exist.

Unless otherwise defined, all technical and scientific terms used herein have the same meanings as those generally understood by those skilled in the art to which the present disclosure belongs. The terms used in the description of the present disclosure are intended to merely describe specific embodiments, but not to limit the present disclosure. The term “and/or” used herein comprises any and all of combinations of one or a plurality of related listed items.

In some embodiments, an ultrasonic probe is provided. The ultrasonic probe may include: a probe body which may be used to ultrasonically scan a target object; an elastic liner surrounding the probe body, where the probe body may pass through the elastic liner and be movable relative to the elastic liner; an adjusting assembly provided with a containing cavity, where the elastic liner may be arranged in the containing cavity and be able to be abutted against a cavity wall of the containing cavity, and where the probe body may be tightened or loosened by the adjusting assembly by tightening or loosening the elastic liner so as to adjust a position of the probe body with respect to the elastic liner; and a fixing member fixedly connected with the adjusting assembly, where the fixing member may be used to be secured to the target object.

In some embodiments, a periphery of the elastic liner may have a drum shape and be abutted against the cavity wall of the containing cavity, and a middle part of the elastic liner may surround the probe body and be abutted against a peripheral wall of the probe body.

In some embodiments, the elastic liner may include two U-shaped clamping members, and end parts of the two U-shaped clamping members may be connected to form the elastic liner.

In some embodiments, the adjusting assembly may include an elastic adjustment base and an adjusting rotary knob sleeved on a periphery of the elastic adjustment base, the elastic adjustment base may be fixedly connected with the fixing member, the containing cavity may be arranged in the elastic adjustment base, and the elastic adjustment base may be squeezed or loosened by adjusting the adjusting rotary knob to squeeze or loosen the elastic liner, thereby adjusting the elastic liner to tighten or loosen the probe body.

In some embodiments, an external thread may be arranged on the periphery of the elastic adjustment base, the adjusting rotary knob may be provided with an internal thread matched with the external thread on the elastic adjustment base, and the elastic liner may be squeezed or loosened by the elastic adjustment base by tightening or loosening the adjusting rotary knob.

In some embodiments, the adjusting assembly may further include a base fixedly connected with the elastic adjustment base, the base may be provided with a through hole through which the probe body may pass, and the base may be fixedly connected with the fixing member.

In some embodiments, the elastic adjustment base may include a plurality of elastic clamping pieces secured to the base, a shape of an inner wall of the elastic clamping piece may be matched with a shape of an outer wall of the elastic liner, an outer wall of the elastic clamping piece may be provided with an external thread, gaps may be arranged between adjacent elastic clamping pieces, and the plurality of elastic clamping pieces may form the containing cavity.

In some embodiments, the base and the fixing member may be fixedly connected by snap, thread or glue, etc.

In some embodiments, the adjusting rotary knob may be a hollow cylinder structure, and a stripe for facilitating handhold or rotation of the adjusting rotary knob may be provided on the periphery of the adjusting rotary knob.

In some embodiments, the fixing member may be a sticking plate or absorbing plate which is able to be secured to a surface of the target object.

In some embodiments, an ultrasonic imaging system is provided. The ultrasonic imaging system may include the ultrasonic probe described above.

In the above ultrasonic probe and the ultrasonic imaging system comprising the ultrasonic probe, the angle of the ultrasonic waves transmitted by the ultrasonic probe and the pressurization depth of the probe body can be adjusted, and the ultrasonic probe can be secured to a target object for a long time for continuous ultrasonic imaging. Therefore, the ultrasonic probe can be widely applied to a clinical occasion where long-time continuous ultrasonic monitoring is required.

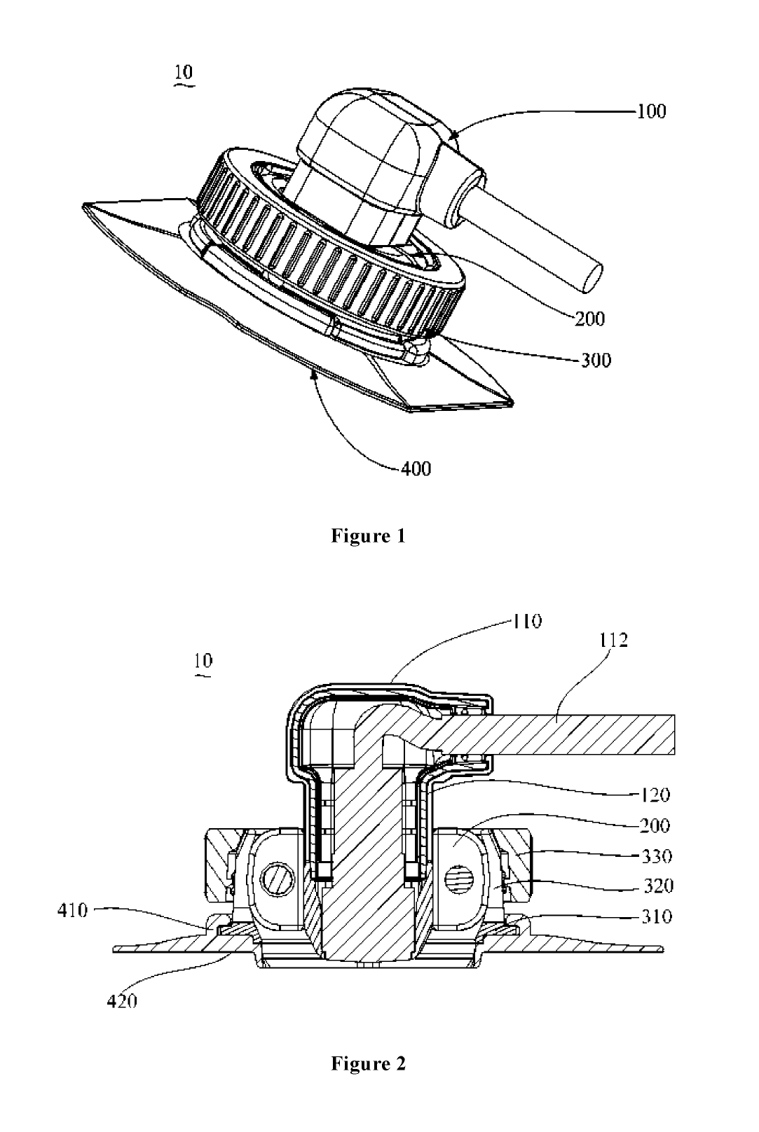

As shown in FIG. 1, an ultrasonic probe 10 of one embodiment may include a probe body 100, an elastic liner 200, an adjusting assembly 300 and a fixing member 400. The ultrasonic probe 10 of the embodiment can be used in an ultrasonic imaging system to perform an ultrasonic imaging on a person to be detected, and especially where a prolonged ultrasonic monitoring is required.

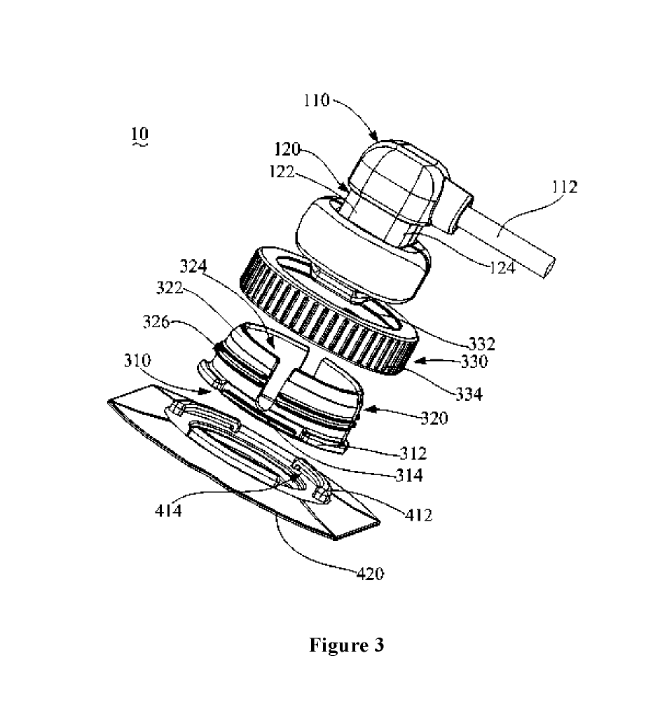

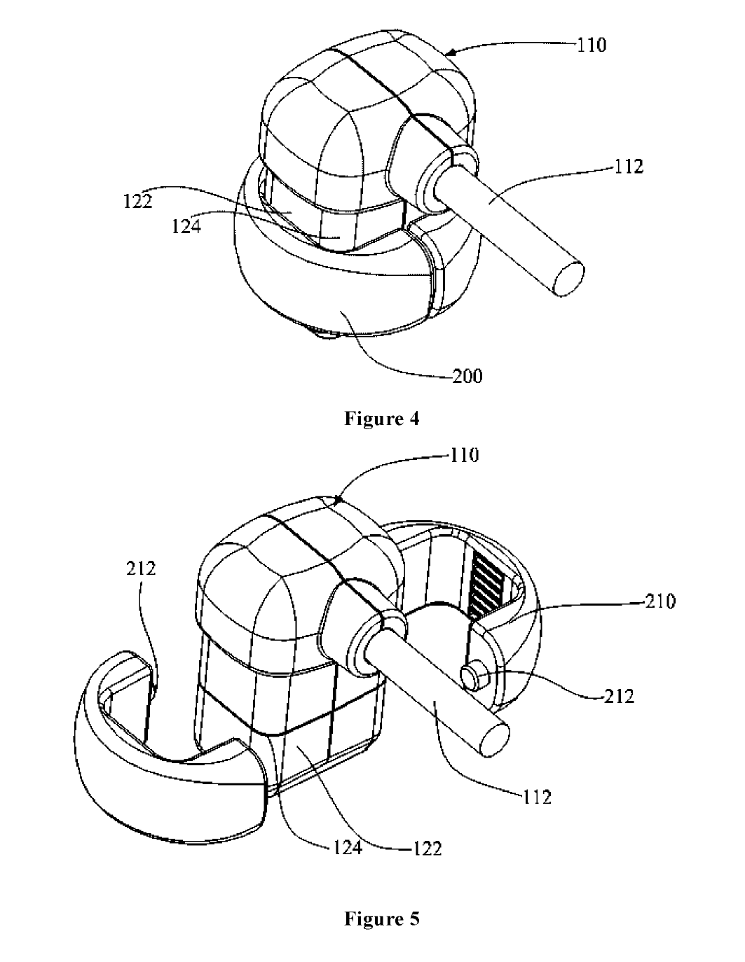

In combination with FIG. 3, FIG. 4 and FIG. 5, the probe body 100, which may be used to image a target object, may include a base part 110 and a detection part 120. A conducting wire 112 may be connected to the base part 110, and elements such as a plate card, cable, etc. (not shown in the drawings) may be arranged in the base part 110. The detection part 120 may be fixedly connected with the base part 110, and elements such as ultrasonic transducers, etc. (not shown in the drawings) may be arranged in the detection part 120. The detection part 120 of the embodiment may have a column shape and comprise four side walls 122. An arc part 124 may be arranged between the side walls 122 to connect adjacent side walls 122, thereby forming a non-rectangular connecting structure which may facilitate the clamping of the elastic liner 200 on the peripheral wall of the whole detection part 120 and the rotation of the detection part 120 in the elastic liner 200 for adjusting the position of the detection part 120 with respect to the elastic liner 200 (for example, adjusting the depth of the detection part 120 in the elastic liner 200 by rotation, etc.).

The elastic liner 200 may be made of elastic material. In combination with FIG. 3 and FIG. 4, the elastic liner 200 of the embodiment may have a flat column shape. The peripheral wall of the elastic liner 200 may be presented as a drum-shaped bulge. A through hole corresponding to the periphery shape of the detection part 120 may be formed in the middle part of the elastic liner 200. The detection part 120 may pass through the through hole and may move along the axial direction of the elastic liner 200. After the detection part 120 passes through the through hole, the elastic liner 200 may surround the detection part 120 and can be abutted against the side walls 122 of the detection part 120. As shown in FIG. 5, in one embodiment, the elastic liner 200 may include two U-shaped clamping members 210. One of two end parts of the clamping members 210 may be provided with a bump 212, and the other may be provided with a groove (not shown in the drawing) matched with the bump 212. The elastic liner 200 may be formed by connecting the ends of the two clamping members 210 around the detection part 120 by inserting the bump 212 into the groove.

It can be understood that in other embodiments, the structure of the elastic liner 200 is not limited to what is described above. For example, the elastic liner may be an all-in-one elastic clamping structure or may also be formed by more than two (for example, three or four) clamping members.

In combination with FIG. 1, FIG. 2 and FIG. 3, an adjusting assembly 300 in an embodiment may include a base 310, an elastic adjustment base 320 and an adjusting rotary knob 330. The elastic adjustment base 320 may be arranged on the base 310, and the adjusting rotary knob 330 may be sleeved on the elastic adjustment base 320.

With reference to FIG. 3, the base 310 in the embodiment may be a circular plate-shaped structure, and a through hole (not shown) through which the detection part 120 may pass may be formed in the middle part. Three first stop blocks 312 and three clamping blocks 314 may be arranged at the base 310 around the through hole thereof. The three first stop blocks 312 and the three clamping blocks 314 may be arranged alternately, and may be evenly distributed around the periphery of the through hole on the base 310. The thickness of the first stop blocks 312 may be greater than that of the clamping blocks 314.

As shown in FIG. 3, the elastic adjustment base 320 in the embodiment may include four elastic clamping pieces 322. One end of the elastic clamping pieces 322 may be secured to the base 310 around the through hole, and the other end may be a free end. The shape of the inner wall of the elastic clamping pieces 322 may be matched with the shape of the outer wall of the elastic liner 200 such that the inner wall and the outer wall can seamlessly abut with each other. Therefore, the four elastic clamping pieces 322 may form a containing cavity 324 which can contain the elastic liner 200. An external thread 326 may be arranged on the outer wall of the elastic clamping piece 322. Gaps may be arranged between adjacent elastic clamping pieces 322, but the external threads 326 on the elastic clamping pieces 322 may be matched with each other to form a discontinuous external thread structure.

In combination with FIG. 1, FIG. 2 and FIG. 3, the adjusting rotary knob 330 in the embodiment may be a hollow cylinder structure. An internal thread 332 matched with the external thread 326 on the elastic adjustment base 320 may be arranged on the inner wall. A stripe 334, which extends along the axial direction of the adjusting rotary knob 330 and facilitates the handhold and rotation of the adjusting rotary knob 330, may be provided on the outer wall of the adjusting rotary knob 330.

After the elastic liner 200 is assembled, it may be placed in the containing cavity 324. The adjusting rotary knob 330 may be sleeved on the periphery of the elastic adjustment base 320, and the detection part 120 may be inserted into the elastic liner 200. After the detection part 120 is inserted into the elastic liner 200 to an appropriate depth, the adjusting rotary knob 330 may be tightened, and the elastic adjustment base 320 may be squeezed by the adjusting rotary knob 330 so as to squeeze the elastic liner 200 to tighten the detection part 120. When the insertion depth of the detection part 120 needs to be adjusted, the adjusting rotary knob 330 may be loosened and the detection part 120 may be adjusted to another appropriate depth, and then the adjusting rotary knob 330 may be tightened again. The operation is easy and simple.

It can be understood that the shapes of the elastic adjustment base 320 and the adjusting rotary knob 330 are not limited to those described above. For example, the number of elastic clamping pieces may also be two or three, etc. The elastic adjustment base 320 may also have a completely hollow column structure, which may be made of elastic material and have an external thread. The elastic adjustment base 320 and the adjusting rotary knob 330 may also be connected in other manners instead of thread connection. For example, the thickness (i.e., the wall thickness of the containing cavity 324) of the elastic adjustment base 320 may gradually increase as it approaches the base 310. The adjusting rotary knob 330 may be directly sleeved on the elastic adjustment base 320. When the adjusting rotary knob 330 is moved towards the base 310, a squeezing force applied on the elastic adjustment base 320 can be gradually increased such that the elastic liner 200 may be elastically squeezed by the elastic adjustment base 320. The stripe 334 may not be provided on the adjusting rotary knob 330, or the stripe 334 may not be limited to the arrangement along the axial direction of the adjusting rotary knob 330 described above, etc. Further, the elastic adjustment base 320 may be directly secured to the fixing member 400 without the base 310.

In combination with FIG. 2 and FIG. 3, the fixing member 400 in the embodiment may be a sticking plate, which may include a fixed part 410 and a sticking part 420. The sticking part 420 may be a sheet plate made of medical polymer material (e.g., thermoplastic polyurethane elastomer plastic (TPU), etc.) and may be used to be stuck to a target object. A through hole through which the detection part 120 may pass may be formed in the middle part of the sticking part 420. The fixed part 410 may be arranged around the periphery of the through hole on the fixing member 400 and may include three second stop blocks 412. The three second stop blocks 412 may be evenly arranged around the through hole, and a notch corresponding to the size of the first stop blocks 312 may be provided between adjacent second stop blocks 412. Clamping grooves 414 may be formed in the second stop blocks 412. The shapes and the sizes of the clamping grooves 414 may be matched with the shapes and the sizes of the clamping blocks 314, such that the clamping blocks 314 can be inserted into the clamping grooves 414. Accordingly, the first stop blocks 312 may be clamped in the notches between two adjacent second stop blocks 412 so as to achieve the connection of the fixing member 400 with the base 310.

It can be understood that in other embodiments, the fixing member 400 can also employ other mechanisms, such as the absorbing plate, etc., which can be secured to the surface of a target object. Moreover, the fixing member 400 and the base 310 can also be fixedly connected by a thread or by other sticking structures, etc. For example, several locating blocks may be arranged on the base 310, locating grooves may be formed in corresponding positions on the fixing member 400, and the locating blocks of the base 310 may be stuck in the locating grooves by glue, etc. Moreover, the number of first stop blocks 312, clamping blocks 314 and second stop blocks 412 may not be limited to those described above, but may also be another appropriate number.

The ultrasonic probe 10 described above can be widely applied to medical devices such as a medical ultrasonic imaging system, ultrasonic diagnostic apparatus, ultrasonic monitor, etc., and may have the advantage that the angle of the ultrasonic waves transmitted by the probe body 100 and the pressurization depth of the probe body 100 can be adjusted. In addition, the ultrasonic probe 10 can be secured to a target object for a long time for continuous ultrasonic imaging, and therefore can be widely applied to a clinical occasion where long-time continuous ultrasonic monitoring is required. Further, an automatic control system can also be added to the ultrasonic probe 10 in the present application to automatically adjust the scanning angle and/or pressurization depth of the probe as needed. Thus, an ultrasonic monitoring device can be further intelligentized. The ultrasonic imaging system in the present application may include the previous ultrasonic probe and a control system, which may include various functional modules or circuits for achieving scanning, reception, signal processing, display, etc.

The above embodiments only present several implementation modes of the present disclosure, and are described in detail. However, it shall not be consequently interpreted as a limitation to the scope of the present disclosure. It should be noted that, for those ordinarily skilled in the art, several modifications and improvements can also be made without departing from the concept of the present disclosure, all of which belong to the protection scope of the present disclosure. Therefore, the protection scope of the present disclosure shall be defined by the appended claims.

We claim:

1. An ultrasonic probe comprising:
a probe body which is used to ultrasonically scan a target object;
an elastic liner surrounding the probe body, wherein the probe body passes through the elastic liner and is movable relative to the elastic liner;
an adjusting assembly provided with a containing cavity, wherein the elastic liner is arranged in the containing cavity and is able to be abutted against a cavity wall of the containing cavity, and the probe body is tightened or loosened by the adjusting assembly by tightening or loosening the elastic liner so as to adjust a position of the probe body with respect to the elastic liner; and
a fixing member fixedly connected to the adjusting assembly, wherein the fixing member is configured to be secured to the target object.

2. The ultrasonic probe of claim 1, wherein a periphery of the elastic liner has a drum shape and is abutted against the cavity wall of the containing cavity, and a middle part of the elastic liner surrounds the probe body and is abutted against a peripheral wall of the probe body.

3. The ultrasonic probe of claim 1, wherein the elastic liner comprises two U-shaped clamping members, and end parts of the two U-shaped clamping members are connected to form the elastic liner.

4. The ultrasonic probe of claim 1, wherein the adjusting assembly comprises an elastic adjustment base and an adjusting rotary knob sleeved on a periphery of the elastic adjustment base, the elastic adjustment base is fixedly connected with the fixing member, the containing cavity is arranged in the elastic adjustment base, and the elastic adjustment base is squeezed or loosened by adjusting the adjusting rotary knob to squeeze or loosen the elastic liner, thereby adjusting the elastic liner to tighten or loosen the probe body.

5. The ultrasonic probe of claim 4, wherein an external thread is arranged on the periphery of the elastic adjustment base, the adjusting rotary knob is provided with an internal thread matched with the external thread on the elastic adjustment base, and the elastic liner is squeezed or loosened by the elastic adjustment base by tightening or loosening the adjusting rotary knob.

6. The ultrasonic probe of claim 4, wherein the adjusting assembly further comprises a base fixedly connected with the elastic adjustment base, the base is provided with a through hole through which the probe body passes, and the base is fixedly connected with the fixing member.

7. The ultrasonic probe of claim 6, wherein the elastic adjustment base comprises a plurality of elastic clamping pieces secured to the base, a shape of an inner wall of the elastic clamping piece is matched with a shape of an outer wall of the elastic liner, an outer wall of the elastic clamping piece is provided with an external thread, gaps are arranged between adjacent elastic clamping pieces, and the plurality of elastic clamping pieces form the containing cavity.

8. The ultrasonic probe of claim 6, wherein the base and the fixing member are fixedly connected by snap, thread or glue.

9. The ultrasonic probe of claim 4, wherein the adjusting rotary knob is a hollow cylinder structure, and a stripe for facilitating handhold or rotation of the adjusting rotary knob is provided on a periphery of the adjusting rotary knob.

10. The ultrasonic probe of claim 1, wherein the fixing member is a sticking plate or absorbing plate which is able to be secured to a surface of the target object.

11. An ultrasonic imaging system, comprising the ultrasonic probe of claim 10.
